(19) United States
(12) Patent Application Publication
(10) Pub. No.: US 2014/0205972 A1
(43) Pub. Date: Jul. 24, 2014

(54) GLASS CERAMIC MATERIAL AND METHOD

(71) Applicant: HARRYSON CONSULTING GMBH, Wilen B. Wollerau (CH)

(72) Inventor: Wei Xia, Uppsala (SE)

(73) Assignee: Harryson Consulting GmbH, Wilen B. Wollerau (CH)

(21) Appl. No.: 13/744,626

(22) Filed: Jan. 18, 2013

(51) Int. Cl. A61K 6/02; A61C 13/00

(52) U.S. Cl. CPC A61K 6/0205; A61C 13/00
USPC 433/215; 501/32; 264/16; 264/621; 501/12; 65/17.2

(57) ABSTRACT

The present invention relates to a method for manufacturing of a glass ceramic material for dental applications. The method comprises: providing a first precursor comprising silicon(IV); providing a second precursor comprising zirconium(IV); hydrolyzing said first precursor and second precursor in solution; polymerizing of the hydrolysed first precursor and second precursor in a solvent, wherein polymers are formed; formation of colloids comprising said polymers; formation of a gel from said colloids; aging the gel; drying the gel; and sintering the gel under formation of a glass ceramic material.

### TECHNICAL FIELD

The present invention relates to methods for manufacturing of a glass ceramic material. The invention further relates to products obtainable by the methods, and to glass ceramic materials obtainable by the methods. The invention further relates to glass ceramic materials and glass ceramic bodies.

### TECHNICAL BACKGROUND

Ceramic- and glass ceramic materials are used as dental materials due to their mechanical properties and aesthetics. The all-ceramic materials, such as zirconia and alumina, have the advantage of high mechanical properties, but are opaque and thus less aesthetically pleasing and more difficult to adapt to the colour of the surrounding teeth than glass ceramic materials such as lithium disilicates, which are translucent. However, the glass ceramic materials generally have a fracture toughness and flexural strength considerably lower than the all-ceramic materials do. A translucent material with improved mechanical properties is desirable for dental restoration applications.

Furthermore, it is desired to produce crack-free samples with desirable mechanical properties having large sizes suitable for large dental works such as bridges, for example samples larger than 1 cm³.

### SUMMARY OF THE INVENTION

Purposes of the present invention include providing solutions to problems identified with regard to prior art.

The present invention allow for efficient manufacturing of a glass ceramic material suitable for, for example, dental applications.

According to a first aspect of the present invention, there is provided a method for manufacturing of a glass ceramic material for dental applications. The method comprises: providing a first precursor comprising silicon(IV); providing a second precursor comprising zirconium(IV); hydrolyzing said first precursor and said second precursor in solution; polymerizing of the hydrolysed first precursor and second precursor in a solvent, wherein polymers are formed; formation of colloids comprising said polymers; formation of a gel from said colloids; aging the gel; drying the gel; and sintering the gel under formation of a glass ceramic material.

Dental applications may be, for example, dental restorations.

A glass ceramic material manufactured in accordance with the first aspect of the invention may comprise nano-sized grains, and particularly nano-sized tetragonal ZrO₂ in a SiO₂ matrix. The glass ceramic material may efficiently be used for dental restorations. The material may be shaped into suitable shapes such as the shape of a tooth, teeth, dental bridges or full arches, for example by fabrication with CAD/CAM processes. The material may have several beneficial properties, for example in the field of dental restoration, such as high translucency and high toughness. The properties make the material particularly suitable for large posterior dental restorations, such as bridges or full-arches, or crowns. Further, large bodies made of the material may be obtained, such that a single body efficiently may be used for manufacturing of several restorations, thus minimizing waste and tool wear.

According to one embodiment, said first precursor may be silicon(IV) alkoxide or silicon(IV) halide, and said second precursor may be zirconium(IV) alkoxide or zirconium(IV)halide.

According to one embodiment, said first precursor may be silicon(IV) alkoxide, and said second precursor may be zirconium(IV) alkoxide.

The silicon(IV) alkoxide may be selected from the group consisting of silicon(IV) methoxide, silicon(IV) ethoxide, silicon(IV) propoxide, and silicon(IV) butoxide, or combinations thereof.

The zircon(IV) alkoxide may be selected from the group consisting of zircon(IV) methoxide, zircon(IV) ethoxide, zircon(IV) propoxide, and zircon(IV) butoxide, or combinations thereof.

According to one embodiment the first precursor is silicon(IV) ethoxide and the second precursor is zircon(IV) propoxide.

According to one embodiment, the halides may be selected from the group comprising fluoride, chloride, bromide or iodide, or combinations thereof. According to one embodiment, it may be preferred that the halide is chloride.

According to one embodiment, the silicon(IV) alkoxide may be selected from the group consisting of tetralkyl orthosilicate and tetraethyl orthosilicate, and the zirconium(IV) alkoxide may be selected from the group consisting of tetralkyl zirconate and tetrapropyl zirconate.

The glass ceramic material may be biocompatible, which makes it particularly suitable to use in certain dental applications.

The method, may be described as comprising a sol-gel method.

According to one embodiment, the polymerizing results in a homogeneous system or a homogeneous polymer.

The aging and/or the drying of the gel may be made such that a continuous polymeric network is obtained or such that discrete particles are obtained. The continuous polymeric network may be a monolith, or a monolithic xerogel. For example, longer drying times, such as one or several weeks, eg. 1 to 4 weeks, may favor formation of the continuous polymeric network while shorter drying times, such as 1 or more days, for example, 2-5 days, or 2-3 days, may favor formation of discrete particles. The drying may take place in room temperature. The discrete particles may have an average size of below 1 micrometer, preferably 10-100 nm and most preferably 10-40 nm, even more preferred 20-40 nm. It is realized that even if a major part by weight of the material is in the form of small discrete particles of such sizes, the material may also contain larger particles or other larger structures.

It may be a benefit with the discrete particles, for example, that they can be produced in a large amount as they do not suffer from the risk of disadvantageous cracks being formed during drying, and that a large amount of discrete particles can be divided into smaller fractions for treatment into bodies of glass ceramic material. Further, a desired shape may be made and sintered, such as by compacting to a desired shape followed by sintering. Thus, machining may be minimized or avoided.

It may be beneficial with the monolithic structure, for example, that a desired shape of the monolith may be made directly for example by casting of the gel in a dye or mould. Thus, machining may be minimized or avoided.

According to one embodiment, the drying the gel may result in discrete particles, or a powder, of the material being formed.

According to one embodiment, the drying the gel may result in a continuous polymeric network of the dried material. Such a material may result in a monolithic structure of the glass ceramic material after sintering.

According to one embodiment, particularly useful for the discrete particles, the dried gel may be compacted into a green body prior to the sintering. According to one embodiment, the compacting is performed at 50 MPa or higher pressures, such as 50 to 500 MPa, or 100 to 200 MPa. Pressing agents, such as PEG or PVA or mixtures thereof, may be mixed with the powder before the pressing in order to improve the pressing. The total amount of PEG and PVA may be 1-20 percent by mass, such as 3-7%, or 5-10%. The compacting may be preceded by grinding, or other means of producing a powder containing to a major part discrete particles.

According to one embodiment, the sintering may be by means of pulsed current directly passing through the gel or particles.

According to one embodiment, the sintering of the gel may be spark plasma sintering. Such sintering may be useful for both the continuous polymeric network and the discrete particles. Other similar or suitable techniques may also be used if suitable, such as techniques known as field assisted sintering (FAST) or pulsed electric current sintering (PECS). According to one embodiment, the material being sintered may be heated at rates of up to 1000 K/min. Such sintering techniques may be particularly efficient for sintering of particles or granules having sizes below 1 micrometer.

According to one embodiment, the sintering may be hot isostatic pressure sintering. Such sintering may be useful for both the continuous polymeric network and the discrete particles.

According to one embodiment, the spark plasma sintering or the hot isostatic pressure sintering may be used for sintering of the material in the shape of a disc or a cube.

According to one embodiment, wherein the drying of the gel results in discrete particles being formed, the sintering is spark plasma sintering or hot isostatic pressure sintering.

According to one embodiment, the sintering may be preceded by heat treatment wherein solvent and/or organic matter is removed from the gel.

The high temperatures and pressures of hot isostatic pressure sintering and/or spark plasma sintering may result in a glass ceramic material with low porosity and small grain sizes, which lends the glass ceramic material excellent properties including a high translucency and high strength. For example, the glass ceramic material and bodies prepared from the glass ceramic material may have a fracture toughness of 2-6 MPa, preferably 2.5-5 MPa, more preferably 3-5 MPa and most preferably 3.5-5 MPa. For example, the translucency may be 70% or higher, such as 70-90%, preferably 70-85%.

According to one embodiment, which may be used with monolithic gels or discrete powders, the sintering may be heating of the dried gel to 900° C. or above, such as 900 to 1200° C., preferably 950 to 1150° C., more preferably 1000 to 1100° C. According to one embodiment, the sintering of the monolithic structure may take up to three weeks, such as one day to three weeks, or 1 week to three weeks.

The sintering may result in nano-sized tetragonal ZrO₂ in a SiO₂ matrix.

According to one embodiment, the method may be for manufacturing glass ceramic material for applications in dental restorations.

The method results in excellent properties of the material such as high strength and high translucency suitable for dental restorations such as bodies in the shape of a part of a tooth, a tooth, or a dental bridge.

According to one embodiment, the method may further comprise forming a dental restoration body from the sintered glass ceramic material.

A dental restoration body, such as a body in the shape of a whole or part of a tooth, a crown, or a dental bridge, produced from the material according to the method of aspects of the invention, may have excellent properties such as high toughness, high translucency, and high strength. For example, the dental restoration body prepared from the glass ceramic material may have a fracture toughness of 2-6 MPa, preferably 2.5-5 MPa, more preferably 3-5 MPa and most preferably 3.5-5 MPa. For example, the translucency may be 70% or higher, such as 70-90%, preferably 70-85%.

According to one embodiment, suitable for the continuous polymeric network, the formation of a gel, the aging of the gel, or the drying of the gel may take place in a dye or mould.

Thus, a desired shape of the gel may efficiently be obtained, and a monolith of that shape may be obtained.

According to one embodiment, the method may further comprise casting of the solution in a dye or mould prior to said formation of a gel.

Thus, a desired shape of the gel may efficiently be obtained.

According to one embodiment, the casting may comprise casting in a dye or mould in a shape selected from the shape of a tooth, teeth, a part of a tooth, or a dental bridge.

Thus, a material suitable for, for example, dental restorations may efficiently be obtained.

According to one embodiment, the sintering may result in a body of glass ceramic material which may be machined to a desired shape.

According to one embodiment, the hydrolysing may take place separately, sequentially or simultaneously.

Thus, for example, the first precursor may be hydrolysed in a first vessel and the second precursor may be hydrolysed in a second vessel; or, the first precursor may first be hydrolysed after which the second precursor is mixed in and hydrolysed, or vice versa; or the first precursor and the second precursor may be hydrolysed at the same time in the same or in different vessels, mixed or non-mixed.

According to one embodiment, the first precursor and the second precursor may be mixed or contacted before hydrolysing.

According to one embodiment, the first precursor may be mixed or contacted with the second precursor prior to hydrolysing. According to one embodiment, the at least partially hydrolysed first precursor may be mixed with the second precursor.

According to one embodiment, the at least partially hydrolysed second precursor may be mixed with the first precursor.

According to one embodiment, the at least partially hydrolysed first precursor may be mixed with the at least partially hydrolysed second precursor.

The hydrolysing takes place in a suitable solvent.

The colloids may be spontaneously formed. The formation of a gel and the aging may take place under removal of liquid or solvent, such as by evaporation. The formation of a gel and aging may, at least partially, take place during drying.

According to one embodiment, the hydrolysing may be performed by means of an added acid, such as a strong acid, for example HCl.

According to one embodiment, the hydrolysis may be partial, to a major extent or complete.

According to one embodiment, particularly suitable for the formation of the continuous polymeric network, the hydrolysis may last from 1 to 24 hours, such hydrolysis may occur at lower reaction rates.

According to one embodiment, particularly suitable for the formation of the discrete particles, the hydrolysis may last from 30 minutes to several hours, for example, 1-5 hours, or 3-4 hours. Such hydrolysis may occur at higher reaction rates.

According to one embodiment, efficient for production of a glass ceramic from the discrete particles, the hydrolysis is complete.

According to one embodiment, the polymerization may be polycondensation.

According to one embodiment, the solution may comprise dimethylformamide. According to one embodiment, the solution may comprise 10-25 mol % of dimethylformamide.

Dimethylformamide may act as drying control additive or a drying agent, which presence may result in improved properties of the glass ceramic material. Further, the presence of dimethylformamide may be useful for minimising the number of cracks in the material. Thus, the glass ceramic material may be stronger and more efficient for use in dental applications. Particularly the monolithic structures may benefit from the use of dimethylformamide, as monoliths free from cracks are desirable,

According to one embodiment, said drying of the gel may comprise: heating in a humid environment; followed by

heating in a dry environment under removal of liquid.

Thus, the gel may be initially heat treated without significant loss of liquid followed by heat treating under drying conditions with loss of liquid.

According to one embodiment, the solvent may comprise a mixture of an alcohol, for example ethanol, and water.

According to one embodiment the first precursor may be provided or hydrolysed in a solvent comprising or to a major part consisting of alcohol, preferably ethanol, such as 95% ethanol.

According to one embodiment the second precursor may be provided or hydrolysed in a solvent comprising or to a major part consisting of alcohol, preferably propanol, more preferably 1-propanol.

According to one embodiment, the glass ceramic material is a ZrO₂—SiO₂ glass ceramic material.

According to one embodiment, 25-50% of the oxides of the glass ceramic material are derived from the second precursor.

According to one embodiment, the formed glass ceramic material may have a translucency of 70% or higher, such as 70-90%, preferably 70-85%.

Such a translucency may be particularly beneficial for dental restorations, for example as it enables the material to be efficiently dyed to a desirable colour of a tooth.

According to one embodiment, the glass ceramic material may comprise grains having an average size below 1 micrometer, preferably 10 to 100 nm, more preferably 20-40 nm.

According to one embodiment, the glass ceramic material may comprise glass ceramic material grains or zirconia comprising grains having an average size below 1 micrometer, preferably 10 to 100 nm, more preferably 20-40 nm.

According to an additional embodiment, the zirconia comprising grains are comprised in a silicon comprising matrix.

According to one embodiment, the molar ratio between the second precursor and the first precursor may be in the range of 20/80 to 60/40, preferably 30/70 to 35/65. According to one embodiment, the molar ratio between zirconium and silicon may be in the range of 20/80 to 60/40, preferably 30/70 to 35/65. According to one example, it may be that a molar ratio between zirconium(IV) alkoxide and silicon(IV) alkoxide may be in the range of 20/80 to 60/40, preferably 30/70 to 35/65.

According to a second aspect, there is provided a method for manufacturing of a glass ceramic material for dental applications, the method comprising: providing a first precursor comprising silicon(IV); providing a second precursor comprising zirconium(IV); hydrolyzing said first precursor and second precursor in solution; polymerizing of the hydrolysed first precursor and second precursor in a solvent, wherein polymers are formed; formation of colloids comprising said polymers; formation of a gel from said colloids; aging the gel; drying the gel such that discrete particles are formed; and sintering the gel under formation of a glass ceramic material.

It is realised that said sintering the gel may be regarded as sintering the particles.

It is further realised that the discrete particles, for example when dried, may be held together in the shape of a solid body, but they do not form a not cross-linked network. They may be treated to, for example, grinding to form a powder of discrete particles.

The drying comprises shorter drying times as compared to drying such that a continuous polymeric network is formed. For example, the drying time may be 1 or more days, for example, 1-5 days, or 2-3 days, which may favor formation of discrete particles. The drying may take place in room temperature.

The formation of discrete particles, may be efficient for producing a glass ceramic material for dental applications, as it enables the particles to be compacted into a desired shape followed by sintering by sintering methods wherein the sizes of the nanoparticles essentially may be maintained resulting in excellent properties of the material.

According to one embodiment, the dried gel may to a major part consist of discrete particles, such as, for example more than 50% per weight.

According to one embodiment, the particles or the grain size of the glass ceramic material may have an average size, for example a diameter, below 1 micrometer, preferably 10 to 100 nm, more preferably 20-40 nm. Such sizes give excellent properties to the glass ceramic material such as high translucency and high strength.

According to one embodiment, the method may comprise prior to said sintering of the gel compacting said particles.

According to one embodiment, said sintering may be spark plasma sintering or hot isostatic pressing sintering, wherein the sintering takes place at or below 1200° C.

According to one embodiment, the gel is subjected to a pressure of 100 to 400 MPa during at least a part of the sintering.

According to one embodiment, the dried gel may be disintegrated into particles or a powder prior to compacting and sintering the gel.

According to one embodiment, the sintering may be performed in less than 60 minutes, such as below 20 minutes or in the range of 1 to 5 minutes.

According to one embodiment, the sintering may be performed at temperatures below 1100° C.

According to one embodiment, the hydrolysis may be essentially complete.

According to one embodiment the method may comprise: providing silicon(IV) alkoxide; providing zirconium(IV) alkoxide or zirconium(IV) chloride; hydrolyzing the silicon(IV) alkoxide, and zirconium(IV) alkoxide or zirconium(IV) chloride, in solution by means of acid; polymerizing of the hydrolysed silicon(IV) alkoxide, and zirconium(IV) alkoxide or zirconium(IV) chloride in a solvent by polycondensation, wherein polymers are formed; formation of colloids comprising said polymers; formation of a gel from said colloids; aging the gel; drying the gel for 1-5 days at room temperature, such that discrete particles are formed having an average size of 10-100 nm; and sintering the gel by means of spark plasma sintering or hot isostatic sintering under formation of a glass ceramic material.

According to a third aspect, there is provided a method for manufacturing of a glass ceramic material for dental applications, the method comprising: providing a first precursor comprising silicon(IV); providing a second precursor comprising zirconium(IV); hydrolyzing said first precursor and second precursor; polymerizing of the hydrolysed first precursor and second precursor, in a solvent, wherein polymers are formed; formation of colloids comprising said polymers; formation of a gel from said colloids; aging the gel; drying the gel such that a continuous polymeric network is formed; and sintering the gel under formation of a glass ceramic material.

The continuous polymeric network may be a monolithic xerogel.

According to one embodiment, the drying may be performed in 4 weeks or less, such as 1 to 4 weeks, or 2 to 3 weeks, preferably at room temperature, in an air atmosphere, such that a continuous polymeric network is formed. The temperature may be, for example, 18-25° C. According to one embodiment, the drying further comprises heat-treating the dried gel at 100° C. in the humidity of a mixture of water and ethanol for 3-12 hours, and then at 140-170° C. in air for 10-24 hours in order to obtain xerogel.

According to one embodiment, the sintering may be performed in 4 days or less, such as 2-3 days.

According to one embodiment, the method may further comprise the step of forming a dental restoration body from the sintered glass ceramic material.

According to one embodiment the method may comprise: providing silicon(IV) alkoxide; providing zirconium(IV) alkoxide or zirconium(IV) chloride; hydrolyzing the silicon(IV) alkoxide, and zirconium(IV) alkoxide or zirconium(IV) chloride by means of acid, in solution; polymerizing of the hydrolysed silicon(IV) alkoxide, and zirconium(IV) alkoxide or zirconium(IV) chloride, in a solvent comprising dimethylformamid, by polycondensation, wherein polymers are formed; formation of colloids comprising said polymers; formation of a gel from said colloids; aging the gel; drying the gel for 1 to 4 weeks at room temperature, such that a continuous polymeric network is formed; and sintering the gel by means of spark plasma sintering or hot isostatic sintering under formation of a glass ceramic material.

According to a fourth aspect, there is provided a product obtainable by the method according to the first, second or third aspects.

According to one embodiment, the product may be used in dental restorations. The glass ceramic material with its properties, for example related to strength and translucency, will lend the product excellent dental properties.

According to a fifth aspect, there is provided a glass ceramic material for dental applications obtainable by the method according to the first, second or third aspects.

According to one embodiment, the glass ceramic material may be used in dental restorations.

According to a sixth aspect, there is provided a glass ceramic material for dental applications comprising ZrO₂—SiO₂, wherein

the molar ratio between ZrO₂ and SiO₂ is in the range of 20/80 to 60/40, preferably 30/70 to 35/65

the fracture toughness of the material is 2-6 MPa, preferably 2.5-5 MPa, more preferably 3-5 MPa, and most preferably 3.5-5 MPa, and

the translucency is 70% or higher, such as 70-90%, preferably 70-85%.

According to one embodiment the glass ceramic material may comprise to a major part ZrO₂—SiO₂. For example, 75-100% or 95-100% of the glass ceramic material may comprise ZrO₂—SiO₂. It may be preferred that the glass ceramic material essentially only consists of ZrO₂—SiO₂.

According to one embodiment, the material comprises grains with an average size below 1 micrometer, preferably 10 to 100 nm, more preferably 20-40 nm.

According to one embodiment, the material may further comprise an additive. For example Li, compounds comprising Li, or Li-ions. According to one embodiment, the material may further comprise 20 m % or less of Li, preferably 1-10 m % of Li.

According to one embodiment, the additive may be at least one colouring agent, or a sintering agent, or mixtures thereof.

According to a seventh aspect, there is provided a glass ceramic body made of the glass ceramic material according to the fifth aspect or the sixth aspect, wherein the body has a volume of at least 1 cm³, for example 1-5 cm³.

According to one embodiment, the body may be in the shape of a disc, a cube, or a rectangular parallelepiped.

According to an eight aspect, there is provided the use of the glass ceramic material according to the fifth aspect or the sixth aspect for dental restorations.
Embodiments and discussions with regard to one aspect may be relevant to one or more of the other aspects. For example embodiments and discussions with regard to the first aspect may be relevant to the second to eighth aspects. References to these embodiments are hereby made, where relevant.

### BRIEF DESCRIPTION OF THE DRAWINGS

FIG. 1 illustrates transmittance versus wavelength for a material produced according to one embodiment.

FIG. 2 illustrates heat treatment and sintering of a xerogel according to one embodiment.

FIG. 3 illustrates heat treatment and sintering of discrete particles according to one embodiment.

FIG. 4 illustrates an XRD pattern of a glass ceramic material according to one embodiment.

FIG. 5 illustrates an XRD pattern of a glass ceramic material according to one embodiment.

### DETAILED DESCRIPTION

The invention will now be explained in more detail, and specific preferred embodiments, and variations of these, will be shown. The explanations are intended for illustrative and explanatory purposes and are not to be seen in any way as limiting the scope of the invention. The illustrations are schematic and all details are not illustrated, and all illustrated details may not be necessary for the invention.

A specific embodiment of the invention will now be discussed. According to this example, a monolithic xerogel is obtained after drying.

ZrO₂—SiO₂ glass ceramics of 30, 35 and 40 mol % ZrO₂ were produced, using a sol-gel method. A drying control additive was used in the present study for reducing the number of cracks in the specimens. Sol-gels were produced using the alkoxide precursors tetraethyl orthosilicate (TEOS) and 70 wt. % tetrapropyl zirconate (TPZ) in 1-propanol (all chemicals were acquired from Sigma-Aldrich, St Louis, Mo., USA). Synthesis was initiated by mixing ethanol (EtOH, >95%), aqueous hydrochloric acid (HCl), for the hydrolysis, and the drying control additive dimethylformamide (DMF) in a 50 ml round bottom flask, followed by the addition of TEOS under continuous stirring. The resulting molar ratio of the solution was 1:1:1:1—TEOS:DMF:EtOH:H2O. The, thus, partially hydrolysed TEOS was magnetically stirred for 3 hours in order to obtain a clear sol and ensure its homogeneity. The desired amount of TPZ was then added slowly using a micropipette and magnetic stirring of the solution was continued overnight. As EtOH is volatile, the sol was kept covered during the stirring in order to minimize any changes in concentration due to evaporation. Depending on the amount of zirconium alkoxide precursor added to the solution, aqueous (0.15 M, 0.40 M or 12.18 M) HCl was then added drop by drop to initiate the final hydrolysis and polymerization of a monolithic gel. After the final synthesis step, the solution was divided and transferred to Teflon® moulds of 25 mm diameter, which were sealed with polymer film for controlled evaporation. The sols were left to gel and age until approximately 40% shrinkage was observed and a stiff gel was formed. In this example, the polymer film was perforated after one week to increase the evaporation after one week which resulted in samples with little or no cracks and samples that were easily detached from the mould. The samples were left to age for approximately three weeks. The use of a hydrophobic mould reduced capillary stresses, which otherwise may result in crack formation during drying.

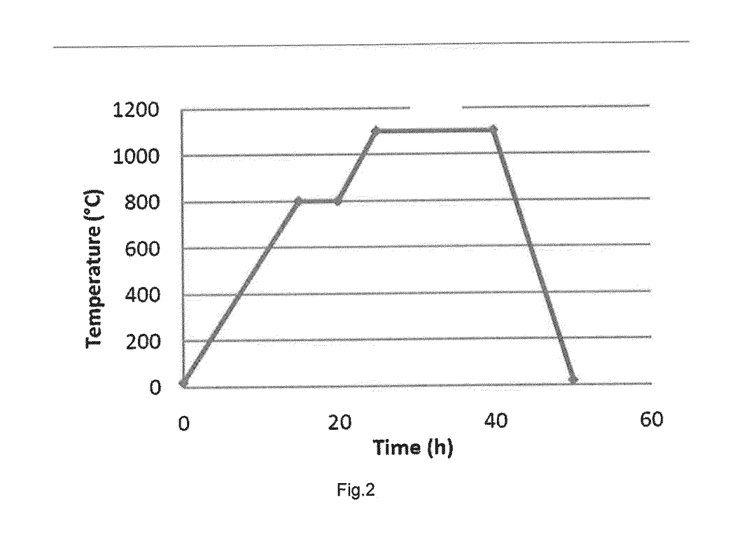

After formation of a stiff xerogel, samples were moved to an oven and kept at 100° C. in an atmosphere of 100% relative humidity for 5 hours. The temperature may be for example 90-110° C.; and the time may be for example 1-10 hours, or 3-6 hours. The temperature was then raised to 150° C. This temperature may be for example 130-170° C., or 140-160° C. and held for 15 hours. This time may be for example 5-20 hours, or 10-20 hours. A subsequent heat treatment process was initiated with a calcination plateau at 800° C. with a holding time of one hour. Sintering of the samples was then carried out at 1100° C., with holding times of 10 or 15 hours. This time may be, for example, 10-15 hours. The ramping rate was 20-30° C./hour. Analysis was performed on samples containing 30, 35 and 40% ZrO₂, sintered at 1100° C. for 10 or 15 h. However, samples containing 40% ZrO₂ were sintered for 10 hour only in this particular example. One example of heat treating and sintering is illustrated in FIG. 2. According to another embodiment, the sintering may be performed using the hot isostatic pressing sintering or spark plasma sintering.

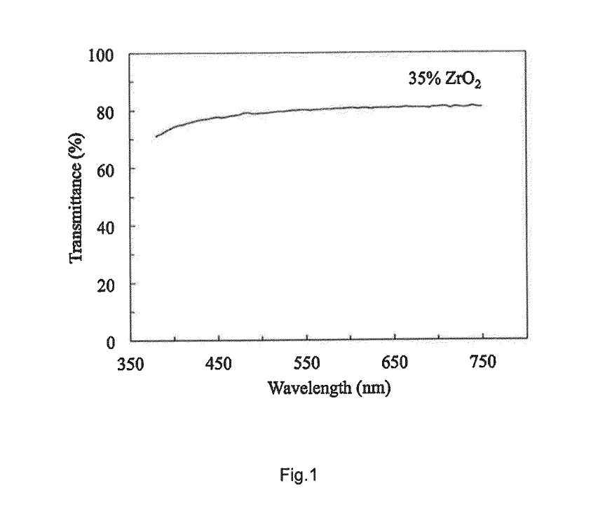

The synthesized materials were evaluated using translucency measurements, X-ray diffraction, nano- and microindentations and corrosion resistance measurements. Transmittance of samples was measured in the visible spectrum (380-750 nm) using a Lambda 900 spectrometer (PerkinElmer, Waltham, Mass., USA) equipped with an integrating sphere detector, coated with Spectralone® (Labsphere Inc., North Sutton, N.H., USA). Transmittance is defined as the ratio of the intensity of transmitted light, I, and the intensity of the incoming light, I₀:T=I/I₀ (1) Measurements were done on samples with a thickness of approximately 1 mm. A sample containing 35% ZrO2 was evaluated. FIG. 1 illustrates that the transmittance over the visible spectra for a sample according to this example containing 35% ZrO₂ is 70% or higher.

The table illustrates crystallite size of tetragonal ZrO₂ nano-grains homogeneously distributed in the silica matrix. The size was calculated from the XRD results using Scherrer's formula of a material in accordance with the specific embodiment.

TABLE 1
Crystallite size of ZrO₂ in monoliths.

| | Crystallite |
|---|---|
| Material | size (nm) |
| 30% ZrO₂—70% SiO₂ | |
| 10 h sintering | 29 |
| 15 h sintering | 33 |
| 35% ZrO₂—65% SiO₂ | |
| 10 h sintering | 28 |
| 15 h sintering | 23 |
| 40% ZrO₂—60% SiO₂ | |
| 10 h sintering | 35 |

A specific embodiment of the invention will now be discussed. According to this example, discrete particles are obtained after drying.

The same sol-gel method as described above with reference to FIGS. 1 and 2 were followed, with the differences that no dimethylformamide was added and with the further differences indicated below.

Drying: The gel was kept for three days at room temperature without cover, and then overnight at 110° C., which removed H₂O and alcohol from the gel.

After synthesis and drying, the obtained material was wet-milled in aqueous solution during 24 hours whereby a fine powder in suspension was obtained. The suspension was filtered and the thus obtained powder was dried to eliminate water. To break up any resulting agglomerates, the material was ground and sieved (50 μm mesh). The powder was then treated by pressing into a tablet in a mould. For each tablet, approximately 0.3 g of powder was prepared into a green piece which suitable dimensions or a thickness of 1 mm and a diameter of 14 mm. Pressing aid Polyvinyl acetate was added. The powder and the pressing aid, 5% by weight, were mixed and pressed under 50 MPa, 7.7 kN, or 10 MPa, 1.5 kN, during 30 seconds.

After the pressing, Cold Isostatic Pressing (CIP) were performed on some samples to increase the green density.

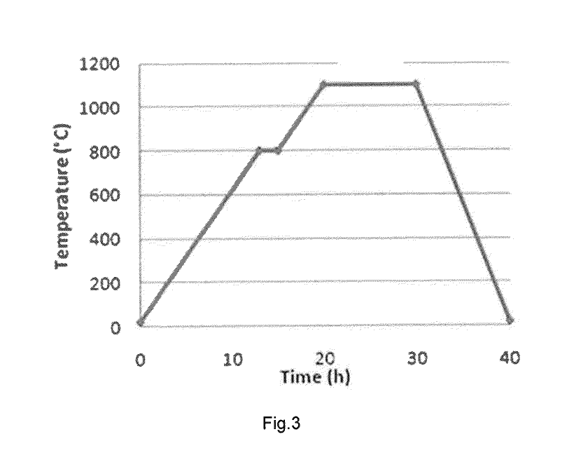

Since the drying was done during the material synthesis, only the sintering is included in the post-preparation heat treatment. A heat program with two levels were used as illustrated in FIG. 3: the calcination, at 800° C. during 1 hour, and the sintering, at 1100° C. during 10 hours. Heating rate between these stages was 60° C./hour. The calcination removes solvents and/or organic components which could be present in material and avoids cracks in sample.

The glass ceramic material had excellent properties.

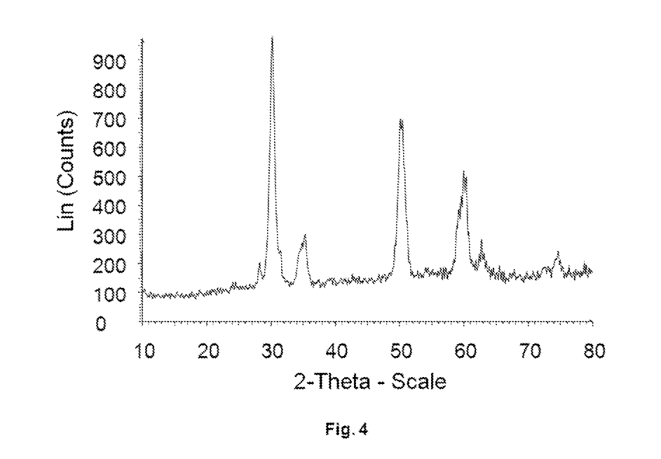

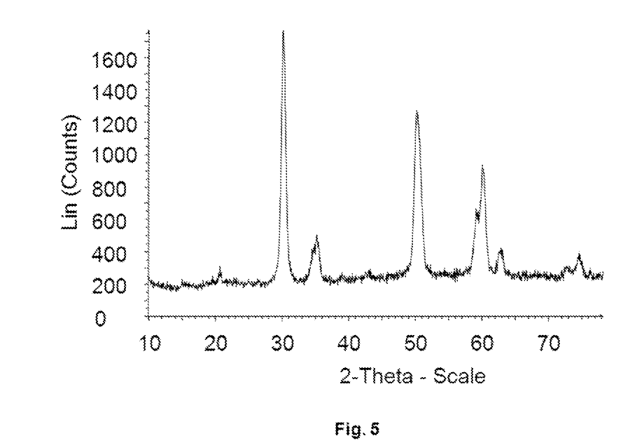

A specific embodiment of the invention will now be discussed with references to FIGS. 4 and 5. According to this example, discrete particles are obtained after drying. The discrete particles were obtained as described with regard to the specific embodiment described above. The sintering for this embodiment was spark plasma sintering. FIGS. 4 and 5, illustrates results from X-ray diffraction analysis of the samples. FIG. 4 illustrates the results of a sample described by having a composition of 50% ZrO₂-50% SiO₂. The peaks are tetragonal ZrO₂. FIG. 5 illustrates the results of a sample described by having a composition of 35% ZrO₂-65% SiO₂. The peaks are tetragonal ZrO₂.

1. A method for manufacturing of a glass ceramic material for dental applications, the method comprising:
providing a first precursor comprising silicon(IV)
providing a second precursor comprising zirconium(IV),
hydrolyzing said first precursor and second precursor in solution,
polymerizing of the hydrolysed first precursor and second precursor in a solvent, wherein polymers are formed,
formation of colloids comprising said polymers
formation of a gel from said colloids,
aging the gel,
drying the gel, and
sintering the gel under formation of a glass ceramic material.

2. The method according to claim 1, wherein
said first precursor is silicon(IV) alkoxide or silicon(IV) halide, and
said second precursor is zirconium(IV) alkoxide or zirconium(IV) halide.

3. The method according to claim 1 or 2, wherein
the first precursor is silicon(IV) alkoxide selected from the group consisting of silicon(IV) methoxide, silicon(IV) ethoxide, silicon(IV) propoxide, and silicon(IV) butoxide, or combinations thereof, and
the second precursor is zirconium(IV) alkoxide selected from the group consisting of zircon(IV) methoxide, zircon(IV) ethoxide, zircon(IV) propoxide, and zircon(IV) butoxide, or combinations thereof.

4. The method according to claim 1, further comprising forming a dental restoration body from the sintered glass ceramic material.

5. The method according to claim 1, wherein the hydrolysing takes place separately, sequentially or simultaneously.

6. The method according to claim 1, wherein the solution comprises 10-25 mol % dimethylformamide.

7. The method according to claim 1, wherein said drying of the gel, comprises:
heating in a humid environment, followed by
heating in a dry environment under removal of liquid.

8. The method according to claim 1, wherein the glass ceramic material has a translucency of 70% or above.

9. The method according to claim 1, wherein the molar ratio between zirconium(IV) and silicon(IV) is in the range of 20/80 to 60/40.

10. The method according to claim 1, wherein said drying the gel is drying the gel such that discrete particles are formed, or drying the gel such that a continuos polymeric network is formed.

11. The method according to claim 1 or 10, further comprising prior to said sintering of the gel
grinding of the dried gel wherein a powder is formed, and
compacting said powder.

12. The method according to claim 1 or 10, wherein said sintering is spark plasma sintering, or hot isostatic pressing,
wherein the sintering takes place at or below 1200° C.

13. The method according to claim 12, wherein the gel is subjected to a pressure of 100 to 400 MPa during the sintering.

14. A method for manufacturing of a glass ceramic material for dental applications, the method comprising:
providing a first precursor comprising silicon(IV)
providing a second precursor comprising zirconium(IV),
hydrolyzing said first precursor and second precursor in solution,
polymerizing of the hydrolysed first precursor and second precursor in a solvent, wherein polymers are formed,
formation of colloids comprising said polymers
formation of a gel from said colloids
aging the gel,
drying the gel such that discrete particles are formed, and
compacting and sintering the gel under formation of a glass ceramic material.

15. The method according to claim 14, wherein the sintering is spark plasma sintering or hot isostatic pressing sintering, wherein the sintering takes place at or below 1200° C.

16. The method according to claim 14, wherein the particles have an average size of below 1 micrometer.

17. A method for manufacturing of a glass ceramic material for dental applications, the method comprising:
providing a first precursor comprising silicon(IV),
providing a second precursor comprising zirconium(IV),
hydrolyzing said first precursor and second precursor,
polymerizing of the hydrolysed first precursor and second precursor in a solvent, wherein polymers are formed,
formation of colloids comprising said polymers
formation of a gel from said colloids,
aging the gel,
drying the gel such that a continuous polymeric network is formed, and
sintering the gel under formation of a glass ceramic material.

18. A glass ceramic material for dental applications comprising ZrO₂—SiO₂, wherein
the molar ratio between ZrO₂ and SiO₂ is in the range of 20/80 to 60/40
the fracture toughness of the material is 2-6 MPa, and
the translucency is 70% or higher.

19. The glass ceramic material for dental applications according to claim 18, the material further comprising an additive.

20. A glass ceramic material for dental applications obtainable by the method according to anyone of claim 1, 14 or 17.

21. The glass ceramic material according to claim 20, used in dental restorations.

22. A use of the glass ceramic material according to claim 18 for dental restorations.

23. A glass ceramic body made of the glass ceramic material according to claim 18, wherein the body has a volume of 1 cm³, or more.

24. A product obtainable by the method according to anyone of claim 1, 14 or 17.
